(19) United States
(12) Patent Application Publication
(10) Pub. No.: US 2022/0343497 A1
(43) Pub. Date: Oct. 27, 2022

(54) Burn severity identification and analysis through three-dimensional surface reconstruction from visible and infrared imagery

(71) Applicant: Sivananthan Laboratories, Inc., Bolingbrook, IL (US)

(72) Inventor: Christopher Frank Buurma, Columbus, OH (US)

(21) Appl. No.: 17/687,310

(22) Filed: Mar. 4, 2022

Related U.S. Application Data
(60) Provisional application No. 63/156,456, filed on Mar. 4, 2021.

(51) Int. Cl. G06T 7/00; H04N 5/33; A61B 5/00; A61B 5/01

(52) U.S. Cl. CPC G06T 7/0012; H04N 5/33; A61B 5/0077; A61B 5/015; A61B 5/6898; A61B 5/445; G06T 2207/10048; G06T 2207/10016; G06T 2207/30088; A61B 2576/00

(57) ABSTRACT

An apparatus and method to assist making treatment decisions for burn injuries. The apparatus will leverage computational imaging methodologies with conventional thermographic analysis techniques. Using infrared sensors, computational image analysis, and burn assessment using thermographic imaging, a complete burn assessment imaging device can be fabricated entirely from commercially-available components. This device will use advanced software paired with a smartphone-mounted infrared camera to perform a detailed thermographic analysis using a burn triage algorithm.

This application claims the benefit of U.S. Provisional Application No. 63/156,456 filed Mar. 4, 2021.

### BACKGROUND

Thermal injuries are common in situations from family households to military conflicts, and care given shortly after a severe burn is critical to treatment and patient recovery. The first several hours and days following a severe burn are most critical, necessitating a rapid triage and treatment plan. Triage for burn injuries is primarily separated into superficial burns that will heal naturally, and deep burns that require surgery.

Non-expert medics usually must make burn triage decisions without the aid of advanced equipment. This is an extreme diagnostic challenge as a clinical assessment from even experienced surgeons can only distinguish between a superficial and a more serious deep burn between 64% and 72% of the time. With inexperienced surgeons this rate drops closer to 50% without the aid of modern technologies. Furthermore, when non-expert medics use the standard thermographic technologies, data interpretation can be exceedingly difficult.

The most common method of burn evaluation is visual observation by a physician. Known methods in burn severity identification have included the use of fluorescent dyes, halogen illumination, and biopsies. U.S. Pat. No. 4,693,255A, hereby incorporated by reference, uses computational analysis of a video recording on the kinetics of tracer dyes to assist a physician in diagnosis. This dye must be injected into the patient prior to computational video analysis by a present physician. The present inventors recognize a desire to make triage decisions in situations where expert personnel and large equipment many not be available.

The present inventors recognize that traditional 2D thermographs of burns is subject to many errors that can distort the size and shape of the burn. These include the camera position, changes in lighting which can affect the analysis through changes in contrast, as well as changes in skin tone and architecture, and the relative position of the burned area on the patient between images. This makes analysis and especially repeated measurement based on 2D imaging difficult to interpret.

The present inventors recognize a desire to assist non-experts in making burn triage decisions without the resources and time available in the clinical setting.

### SUMMARY

Disclosed is an apparatus and method to assist making treatment decisions for burn injuries.

The apparatus will leverage computational imaging methodologies with conventional thermographic analysis techniques. Using infrared sensors, computational image analysis, and burn assessment using thermographic imaging, a complete burn assessment imaging device can be fabricated entirely from commercially-available components. This device will use advanced software paired with a smartphone-mounted infrared camera to perform a detailed thermographic analysis using a burn triage algorithm.

A 3D thermographic model of the burn surface is computationally generated from the video sequences and then is automatically analyzed by a burn triage algorithm which rapidly provides triage recommendations and estimations of the burn depth to non-experts without the need of user input or training.

Compared to a standard thermographic analysis, a computationally generated 3D model of the burn surface can account for many sources of error and greatly compliment an automated analysis. By generating an accurate 3D model from measurements and applying computational analysis methods such as machine vision, image recognition, statistical modeling, thermal volume modeling, and machine learning, many errors can be eliminated or greatly reduced allowing burn triage predictions to be made more accurately, rapidly, and without the need of a trained surgeon to be present. These techniques can also assist a trained surgeon in treatment plans and the monitoring of patient recovery where the initial models can be refined by medical expertise and other analysis methods.

Estimation of burn depth and triage levels can be based on a meta-analysis of existing literature, and utilize the complete 3D surface model of each burn, not just their 2D projections from the image.

The current burn triage model improves the methodologies previously employed for thermographic analysis of burn injuries. First, the injury will be imaged and mapped as a 3D surface and various predictions made of the burn depth and thermal volume. Next, calculations are made of the burn area using machine vision methods (edge and contour detection with thresholding). This burn area will be of the 3D surface, and not from simple 2D images. Following the calculation of burn area, a temperature analysis is completed. This provides more metrics of burn damage: the absolute temperature of the burn overall and at its center, the spatial relative temperature following approximately the three Jackson zones of injury obtained statistically by their distribution, and the change in temperature over time through multiple scans of the same burn which has predictive power in the healing potential and damage extent. The temperature analysis is performed statistically and gives a relative percent of the body that is burned, the percent of each zone of injury, and the statistical mean and standard deviation of temperature changes in space and time.

Each metric from this large set of data yields a Triage Level value from κ to 1, where 0 indicates very low healing potential from this metric indicative of necrosis and the need for surgery, and 1 indicates high healing potential through a likely more superficial burn. At present, a composite score from the different metrics is computed as a simple average. This value can then be mapped for each burn, and can be mapped over the 3D model and related 2D images, giving an easy color-code for the extent of burn damage in a given area. This composite score will then be summed over the detected burn areas and an overall triage recommendation given to the user indicating the potential for healing spontaneously in 21 days, or indicating the need for surgical intervention for proper healing of the injury.

Using this technique, the first responder or combat medic can quickly assess the burn damage via the automated analysis, or they may inspect and analyze the 2D or 3D underlying triage recommendation data themselves. As more data is collected with this system through animal studies or clinical trials, the automated algorithm can be further improved by a variety of means, including a new method of creating a composite score such as a weighted average, creation of new burn damage metrics with more predictive power, or through machine learning methods via a simple neural network classifier from the entire dataset.

Thus, a burn triage recommendation can be made immediately following a scan, with increasing confidence in the recommendation by adding repeated scans during recovery. Scans themselves take less than 10 seconds, with data processing on the order of minutes, allowing for very rapid triage when needed. Since this data can also be captured by a non-professional, scans can be made routinely on patients under care by any available personnel. By providing repeated scans over several hours to days after injury, it is anticipated that the triage recommendation can be made with increasing confidence and accuracy as more data becomes available for the automated routine to analyze. This provides combat medics and first responders with a much earlier prediction of burn healing potential without the need for a surgeon's analysis.

In some embodiments, a commercially available infrared camera can be used in coordination with a commercially available smartphone. A software package for performing triage calculations can be pre-installed on the smartphone. A user of the device would use it to examine a burn injury on a victim. The user positions the smartphone-camera apparatus to capture an image of the victim's burn, and then the user moves the apparatus around in a small arc to capture images of the burn from different angles and positions. After the user has collected sufficient visual data of the burn injury, the images from the visible light camera are input to an algorithm to reconstruct a 3D surface of the burn injury. The processing of this algorithm may occur external to the smartphone device, such as on a desktop computer. The data collected by the infrared camera is then combined with the 3D surface reconstruction to create a 3D thermal surface. The 3D thermal surface is run through several computer vision algorithms to obtain metrics and statistics about the surface.

Several decision heuristics were created from the burn treatment literature. The metrics calculated above are input to the decision heuristics, and each of the heuristics gives a score between 0 and 1. Statistics are computed on the heuristic scores, and the resulting statistics determine the triage decision of whether the burn requires surgery or if it should heal on its own.

Compared to a standard 2D thermographic analysis, a computationally generated 3D model of the burn surface can account for many sources of error and greatly compliment an automated analysis. By generating an accurate 3D model from measurements and applying computational analysis methods such as machine vision, image recognition, statistical modeling, thermal volume modeling, and machine learning, many errors can be eliminated or greatly reduced allowing burn triage predictions made more accurately, rapidly, and without the need of a trained surgeon to be present.

In some embodiments the triage program would also present an intuitive and straightforward interface to the user. The interface navigation may be designed to minimize the need for expertise of the user and demonstrate clearly all actions needed to collect proper data.

Numerous other advantages and features of the present invention will be become readily apparent from the following detailed description of the invention and the embodiments thereof, and from the accompanying drawings.

### BRIEF DESCRIPTION OF THE DRAWINGS

FIG. 1 is a method step flow diagram that shows the process for burn injury triage.

FIG. 2A and FIG. 2B are screen shots from a burn triage software showing exemplary output from the burn triage software from two readings, demonstrating how triage recommendations can change with later observation.

FIG. 3 is an exemplary schematic view of the graphical user interface (GUI) that might be used for the application.

FIG. 4A is a typical screen image captured by an IR camera.

FIG. 4B is a rear perspective view of an exemplary embodiment burn scanning apparatus according to the invention.

FIG. 4C is a front perspective view of the exemplary embodiment burn scanning apparatus of FIG. 4B.

FIG. 5A is a photographic perspective view of an overlay of the IR and visible images of a chilled burn area on a subject's arm showing a clear IR signature.

FIG. 5B is an enlarged photographic perspective view taken from FIG. 5A showing a plotting line drawn on the IR image of chilled skin.

FIG. 5C is a plot of the relative intensity vs distance along the chilled area.

### DETAILED DESCRIPTION

While this invention is susceptible of embodiment in many different forms, there are shown in the drawings, and will be described herein in detail, specific embodiments thereof with the understanding that the present disclosure is to be considered as an exemplification of the principles of the invention and is not intended to limit the invention to the specific embodiments illustrated.

This application incorporates by reference U.S. Provisional Application No. 63/156,456 filed Mar. 4, 2021 in its entirety.

A burn triage procedure is described herein. First, the injury will be imaged and mapped as a 3D surface and various predictions made of the burn depth and thermal volume. Next, calculations are made of the burn area using machine vision methods (edge and contour detection with thresholding). This burn area will be of the 3D surface, and not from simple 2D images. Following the calculation of burn area, a temperature analysis is completed. This provides more metrics of burn damage: the absolute temperature of the burn overall and at its center, the spatial relative temperature following approximately the three Jackson zones of injury obtained statistically by their distribution, and the change in temperature over time through multiple scans of the same burn which has predictive power in the healing potential and damage extent. The temperature analysis is performed statistically and gives a relative percent of the body that is burned, the percent of each zone of injury, and the statistical mean and standard deviation of temperature changes in space and time.

Each metric from this large set of data yields a Triage Level value from 0 to 1, where 0 indicates very low healing potential from this metric, indicative of necrosis and the need for surgery, and 1 indicates high healing potential through a likely more superficial burn. At present, a composite score from the different metrics is computed as a simple average. This value can then be mapped for each burn, and can be mapped over the 3D model and related 2D images, giving an easy color-code for the extent of burn damage in a given area. This composite score will then be summed over the detected burn areas and an overall triage recommendation given to the user indicating the potential for healing spontaneously in 21 days, or indicating the need for surgical intervention for proper healing of the injury. This rather simplistic approach could be enhanced and validated using future clinical studies. Calculation of some example metrics are listed below.

$$T {L}_{a b s T} = \{ \begin{matrix}1 if {T}_{c e nter} > 3 {3}^{∘} C . \\ 0 if {T}_{c e nter} < 3 {1}^{∘} C . \\ \frac{( {T}_{c e nter} - 31 )}{2} otherwise\end{matrix} T {L}_{\partial T / \partial x} = \{ \begin{matrix}1 if {T}_{h e a l t h y} - {T}_{c e nter} \leq {0}^{∘} C . \\ 0 if {T}_{h e a l t h y} - {T}_{c e nter} > {3}^{∘} C . \\ \frac{( {T}_{h e a l t h y} - {T}_{c e nter} )}{3} otherwise\end{matrix} T {L}_{Z o n e s i z e} = \{ \begin{matrix}1 if {A}_{> 0.25 {T}_{max}} / {A}_{t o t} \leq 30 \% \\ 0 if {A}_{> 0.25 {T}_{max}} / {A}_{t o t} > 60 \% \\ 2 - \frac{{A}_{> 0.25 {T}_{max}} / {A}_{t o t}}{0.3} otherwise\end{matrix} T L = \frac{1}{N} {\sum }_{0}^{N} T {L}_{N}$$

Using this technique, the first responder or combat medic can quickly assess the burn damage via the automated analysis, or they may inspect and analyze the 2D or 3D triage recommendation data themselves. As more data is collected with this system through animal studies or clinical trials, the automated algorithm can be further improved by a variety of means, including a new method of creating a composite score such as a weighted average, creation of new burn damage metrics with more predictive power, or through machine learning methods via a simple neural network classifier from the entire dataset.

Thus, a burn triage recommendation can be made immediately following a scan, with increasing confidence in the recommendation by adding repeated scans during recovery. Scans themselves take less than 10 seconds, with data processing on the order of minutes, allowing for very rapid triage when needed.

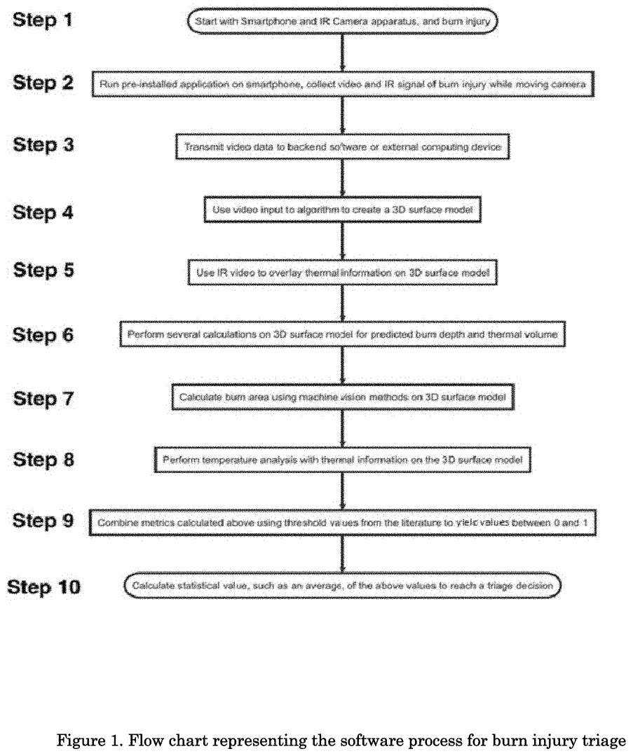

FIG. 1 is an exemplary flow diagram that shows the software-implemented process for burn injury triage.

Step 1: Start with Smartphone and IR camera apparatus, and a burn injury site.

Step 2: Run pre-installed application on smartphone, collect visible light video and IR signal video of burn injury while moving the cameras.

Step 3: Transmit video data to backend software or an external computing device.

Step 4: Use video input to an algorithm to create a 3D surface model.

Step 5: Use IR video to overlay thermal information on 3D surface model.

Step 6: Perform several calculations on 3D surface model for predicted burn depth and thermal volume.

Step 7: Calculate burn area using machine vision methods on 3D surface model.

Step 8: Perform temperature analysis with thermal information on the 3D surface model.

Step 9: Combine metrics calculated above using threshold values from the literature to yield values between 0 and 1.

Step 10: Calculate statistical value, such as an average, of the above values to reach a triage decision.

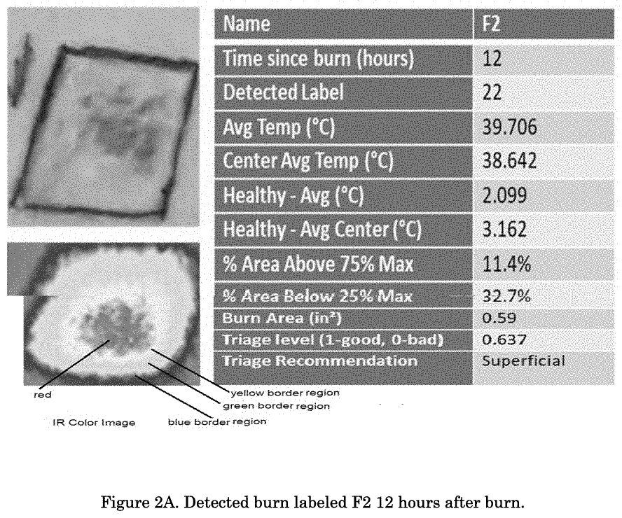

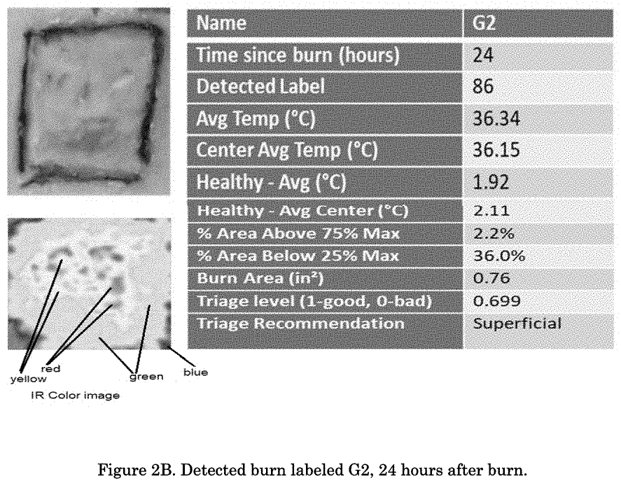

FIG. 2A and FIG. 2B are screen shots from a burn triage software showing exemplary output from the burn triage software from two scans, demonstrating how triage recommendations can change with later observation. Both were observed on the 2^{nd} day of observation. The change in temperature from healthy to the center reduced from Δ3.1° C. to Δ2.1° C., and subsequently the triage level rose from 0.63 to 0.7.

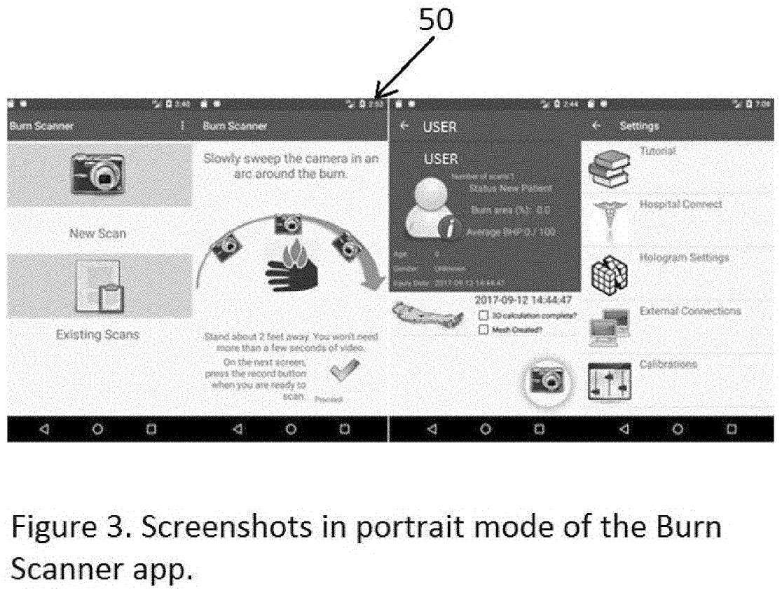

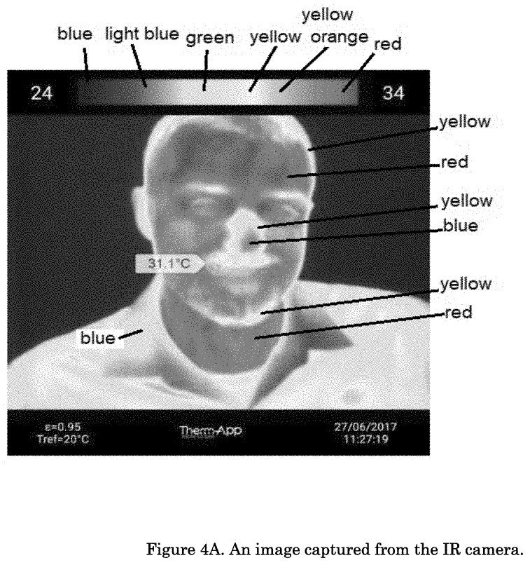

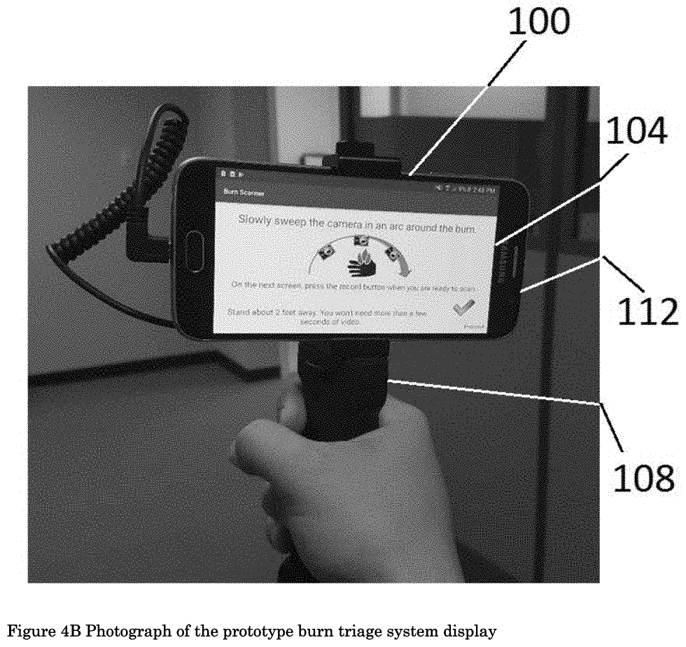

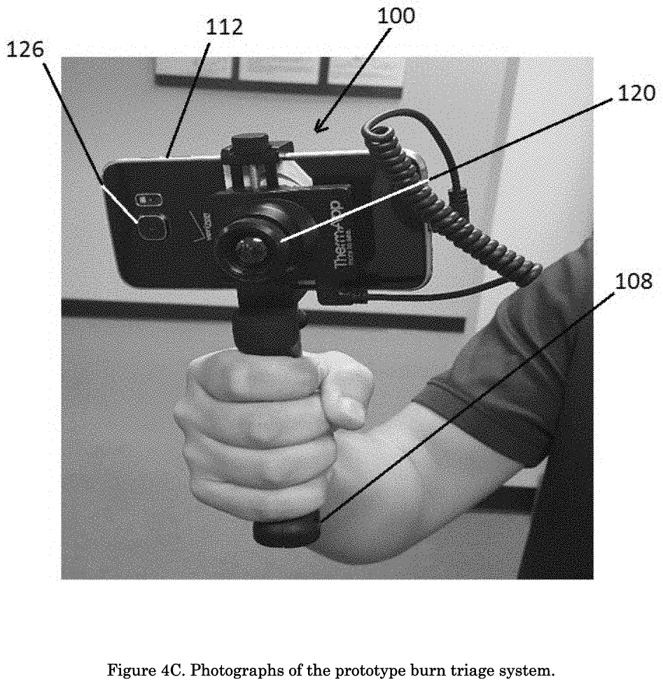

FIG. 3 is an exemplary schematic view of a graphical user interface (GUI) 50 that might be used for the software application. The GUI 50 provides a display of instructions, controls and calibrations. It can be provided on a desktop or other computer or on the device shown in FIGS. 4B and 4C.

FIG. 4A is a typical screen image captured by an IR camera providing a thermal image.

FIG. 4B is a rear perspective view of an exemplary embodiment burn scanning apparatus 100 according to the invention. The apparatus 100 includes a screen 104 mounted on a handle 108. The screen 104 can be part of a smart phone 112.

FIG. 4C is a front perspective view of the exemplary embodiment burn scanning apparatus 100 of FIG. 4B. An IR camera 120 is mounted on the handle 108. A camera 126 for capturing visible images and videos can be provided, such as being provided on the smart phone 112.

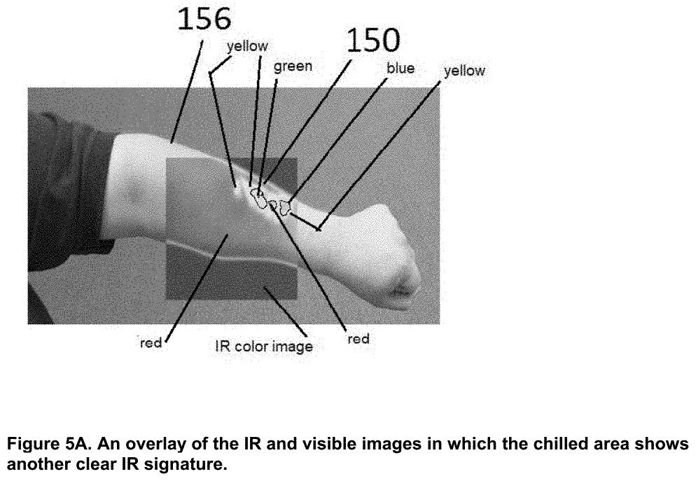

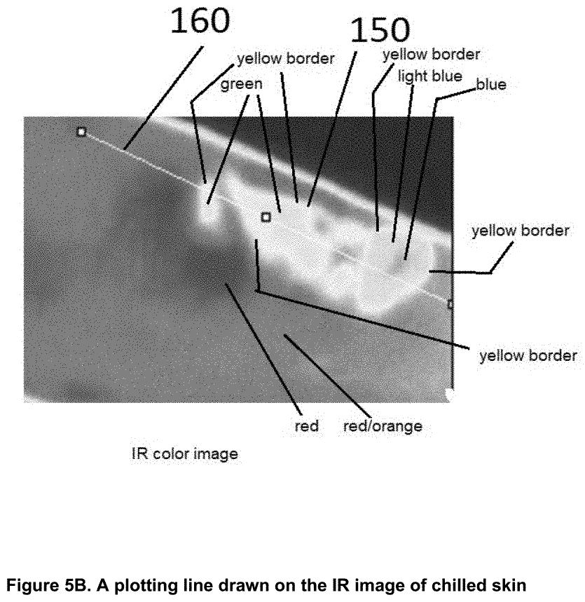

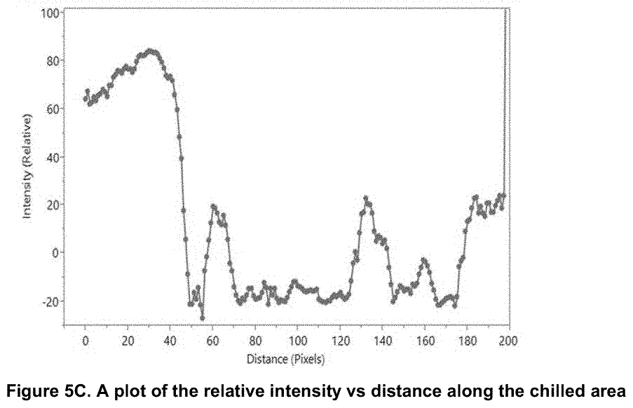

FIG. 5A is a photographic perspective view of an overlay of the IR signature captured by the IR camera 120 and visible images captured by the camera 126, of a chilled burn area 150 on a subject's arm 156 showing a clear IR signature.

FIG. 5B is an enlarged photographic perspective view of the chilled burn area 150 taken from FIG. 5A showing a plotting line 160 drawn on the IR image of chilled skin.

FIG. 5C is a plot of the relative intensity vs distance along the plotting line 160 of the chilled area 150.

From the foregoing, it will be observed that numerous variations and modifications may be effected without departing from the spirit and scope of the invention. It is to be understood that no limitation with respect to the specific apparatus illustrated herein is intended or should be inferred.

The invention claimed is:

1. An apparatus for analyzing burn injuries, comprising:
a smartphone having a visible light camera;
an IR camera;
the smartphone collecting visible light video and IR signal video of a burn injury while moving the cameras;
a computing device using the video to create a 3D surface model;
the computing device overlaying thermal information from the IR camera onto the 3D surface model;
the computing device calculating burn area using machine vision methods on the 3D surface model and analyzing temperature with thermal information on the 3D surface model.

2. The apparatus according to claim 2, wherein the computing device is remote from the smartphone.

3. The apparatus according to claim 1, wherein the computing device is provided within the smartphone.

4. A method for analyzing burn injuries, comprising the steps of:
using a smartphone having a visible light camera and an IR camera, running a pre-installed application on the smartphone to collect video and IR signal video of burn injury while moving the cameras;
transmitting video data to backend software or an external computing device;
using video input to an algorithm to create a 3D surface model;
using IR video to overlay thermal information on the 3D surface model;
performing calculations on the 3D surface model for predicted burn depth and thermal volume;
calculating burn area using machine vision methods on the 3D surface model;
performing temperature analysis with thermal information on the 3D surface model;
combining metrics calculated above using threshold values to yield values between 0 and 1;
calculating a statistical value, such as an average, of the above values to reach a triage decision.
